(19) United States
(12) Patent Application Publication
(10) Pub. No.: US 2010/0292320 A1
(43) Pub. Date: Nov. 18, 2010

(54) BENZOFURAN ANILIDE HISTONE DEACETYLASE INHIBITORS

(75) Inventor: Lawrence S. Melvin, JR., Longmont, CO (US)

Correspondence Address: GILEAD SCIENCES INC, 333 LAKESIDE DR, FOSTER CITY, CA, 94404, US

(73) Assignee: Gilead Colorado, Inc., Foster City, CA (US)

(21) Appl. No.: 12/747,159

(22) PCT Filed: Dec. 12, 2008
(86) PCT No.: PCT/US08/86643

§ 371 Date: Jul. 29, 2010

Related U.S. Application Data
(60) Provisional application No. 61/013,794, filed on Dec. 14, 2007.

(51) Int. Cl. A61K 31/343; C07D 307/00; A61P 35/04; A61P 37/00; A61P 25/28; A61P 25/00; A61P 9/00; A61P 3/10

(52) U.S. Cl. 514/469; 549/467

(57) ABSTRACT

The present disclosure provides a compound of general Formula (I) having enzyme inhibitory activity, a pharmaceutical composition comprising the compound, and a method useful to treat diseases using the compound.

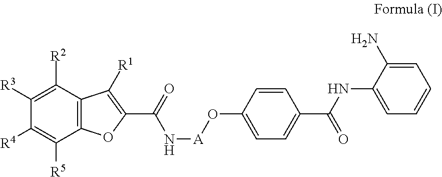

### CROSS REFERENCE TO RELATED APPLICATIONS

This application claims the benefit of U.S. Provisional Application Ser. No. 61/013,794 filed Dec. 14, 2007. The disclosure of the application is hereby incorporated by reference.

### FIELD

The present disclosure generally relates to a compound having enzyme inhibitory activity, pharmaceutical compositions comprising the compound, and methods useful for treating diseases.

### BACKGROUND

Histones are protein components making up chromatin in association with DNA. Histones are subject to covalent modifications of various enzymes such as, for example, histone deacetylase (HDAC), histone methyltransferase (HMT) and histone acetyltransferase (HAT). Covalent modifications of core histones influence protein-protein interaction and protein access to DNA.

HDACs catalyze deacetylation of lysine residues on histones and other proteins. It is known that low levels of histone-acetylation are associated with repression of gene expression. Therefore, abnormal HDAC activities could destroy the delicate balance in cell regulation. The HDACs belong to four structurally and functionally different phylogenetic classes: class I (HDAC-1, -2, -3, and -8) compounds are closely related to yeast RPD3; class IIa (HDAC-4, -5, -7, and -9) and class IIb (HDAC-6 and -10) share domains with yeast HDAC-1; class IV, recently described (comprising HDAC-11), exhibits properties of both class I and class II HDACs. All the above HDACs are zinc dependent proteases. Class III HDACs have been identified on the basis of sequence similarity with Sir2, a yeast transcription repressor, and require the cofactor NAD for their deacetylase function. See, for example, Marielle Paris et al., Histone Deacetylase Inhibitors: From Bench to Clinic, JOURNAL OF MEDICINAL CHEMISTRY 51(11): 3330-3330 (2008).

It has been reported that HDAC activities play an important role in a variety of human disease states. Accordingly, an HDAC inhibitor can provide therapeutic benefits to a broad range of patients. Due to the therapeutic significance, various types of HDAC inhibitors have been developed to date. See, for example, Moradeli et al., Histone Deacetylase Inhibitors: Latest Developments, Trends, and Prospects, CURR. MED. CHEM.: ANTI-CANCER AGENTS 5(5):529-560 (2005).

Ortho-amino-anilide derivatives have been designed and synthesized in an effort to inhibit HDAC activities (see Moradeli et al.).

WO 2004/035525 (Methylgene) mentions ortho-amino anilides linked to a substituted or unsubstituted aryl or heteroaryl group through an alkenyl linker containing a C═C double bond. An example is N-(2-amino-phenyl)-4-{2-[2-(1H-indol-3-yl)-ethylcarbamoyl]-vinyl}-benzamide.

WO 2004/069803 (Hoffmann-La Roche) shows a mono-acylated O-phenylendiamine derivatives which are substituted with various aryl linkers such as thiophen-2,5-diyl, pyridine-2,5-diyl, pyridine-5,2-diyl, pyridine-2,6-diyl, pyridine-2,4-diyl and 1,4-phenylene.

Roberto R. Rosato et al, CANCER RESEARCH 63: 3637-3645 (2003) discusses HDAC inhibitory activity of an ortho-amino anilide compound, N-(2-aminophenyl)-4-[N-(pyridine-3-ylmethoxy-carbonyl)aminomethyl]benzamide (MS-275), when examined in human leukemia, lymphoma cells and primary acute myelogenous leukemia blasts.

Hydroxamate type compounds are another class of HDAC inhibitors that have been researched in clinical settings. WO 2004/092115 mentions a generic family of hydroxamate compounds with a variety of aryl substitutions which fall within the aromatic hydroxamate class, one sub-generic compound of which is directed to hydroxamates with a benzofuran substitution.

In an investigation of a hydroxamate-based series of compounds, it was found that the hydroxamic acid functionality was essential for potency because replacement with a carboxylic acid led to an inactive compound (see Joseph J. Buggy et al, CRA-024781: a novel synthetic inhibitor of histone deacetylase enzymes with antitumor activity in vitro and in vivo, MOL. CANCER THER. 5:1309-1317 (2006)).

### SUMMARY

In various embodiments, there is provided a compound having enzyme inhibitory activity, a composition comprising the compound, and a method useful to treat diseases arising from abnormal cell proliferation or differentiation.

Specifically, the present disclosure is directed to a novel compound of Formula (I) or a pharmaceutically acceptable salt thereof:

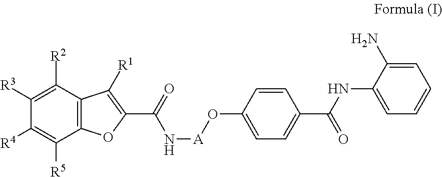

wherein R¹ is selected from the group consisting of H, alkyl, cycloalkyl, alkoxy, amino, aminoalkyl and aryl; and if R¹ is not H, R¹is optionally substituted with halo, hydroxyl, alkyl, cycloalkyl, alkoxy, amino, aryl, haloaryl, heteroaryl or heterocycloalkyl;

R², R³, R⁴ and R⁵ are independently selected from the group consisting of H, alkyl, cycloalkyl, alkoxy, amino, aminoalkyl, aminoalkoxy, carboxyalkylaminoalkyl and aryl; and if any of R², R³, R⁴ and R⁵ is not H, then each of R², R³, R⁴ and R⁵ is optionally substituted with halo, hydroxyl, alkyl, cycloalkyl, alkoxy, amino, aryl, haloaryl, heteroaryl or heterocycloalkyl; and

A is C₂-C₆ alkylenyl optionally substituted with hydroxyl or alkyl, or C₄-C₆ alkenylenyl optionally substituted with alkyl.

In an embodiment, there is provided a pharmaceutical composition comprising an HDAC-inhibitory effective amount of one or more compounds described above and a pharmaceutically-acceptable carrier.

In another embodiment, there is provided a method of inhibiting or treating diseases arising from abnormal cell proliferation and differentiation in animal, comprising administering to said animal a therapeutically effective amount of one or more compounds described above.

The compounds above are more fully described in the detailed description that follows.

### DETAILED DESCRIPTION

The following description is merely exemplary in nature and is not intended to limit the present disclosure, application, or uses.

Definitions

“Alkenylenyl” refers to a linear or branched, unsaturated divalent C₄-C₆ hydrocarbon linker. Examples of alkenylenyl groups include, but are not limited to, butylenylenyl(—CH₂CH═CHCH₂—) and pentylenylenyl (—CH₂CH₂CH═CHCH₂—).

“Alkoxy” is RO— where R is alkyl. Non-limiting examples of alkoxy groups include methoxy, ethoxy and propoxy.

“Alkoxyalkyl” refers to an alkyl moiety substituted with an alkoxy group. Examples of alkoxyalkyl groups include methoxymethyl, methoxyethyl, methoxypropyl and ethoxyethyl.

“Alkoxyalkoxyalkyl” refers to an alkoxyalkyl group substituted with an alkoxy group, wherein alkoxy and alkoxyalkyl are as defined herein. Examples of alkoxyalkoxyalkyl groups include, but are not limited to, methoxyethoxymethyl and ethoxyethoxymethyl.

“Alkyl” refers to a straight or branched chain hydrocarbyl group. In an embodiment, alkyl has from 1 to 12 carbon atoms. In some embodiments, alkyl is a C₁-C₁₀ alkyl group or a C₁-C₆ alkyl group. Examples of alkyl groups include, but are not limited to, methyl, ethyl, propyl, isopropyl, butyl, isobutyl, t-butyl, pentyl, hexyl, heptyl, octyl, nonyl and decyl.

“Alkylamino” refers to an amino group substituted with one or more alkyl groups. “N-(alkyl)amino” is RNH— and “N,N-(alkyl)₂-amino” is R₂N—, where the R groups are alkyl as defined herein and are the same or different. In various embodiments, R is a C₁-C₁₀ alkyl group or a C₁-C₆ alkyl group. Examples of alkylamino groups include methylamino, ethylamino, propylamino, butylamino, dimethylamino, diethylamino, and methylethylamino.

“Alkylaminoalkyl” refers to an alkyl moiety substituted with an alkylamino group, wherein alkylamino is as defined herein. Examples of alkylaminoalkyl groups include methylaminomethyl and ethylaminomethyl.

“Alkylenyl” refers to a linear or branched, saturated divalent C₁-C₆ hydrocarbon linker. Examples of alkylenyl groups include, but are not limited to, ethylenyl (—CH₂CH₂—) and propylenyl (—CH₂CH₂CH₂—).

“Aryl” refers to any monocyclic or bicyclic carbon ring of up to 7 atoms in each ring, wherein at least one ring is aromatic. Aryl encompasses a ring system of up to 14 carbons atoms that includes a carbocyclic aromatic group fused with a 5- or 6-membered cycloalkyl group. Examples of aryl groups include, but are not limited to, phenyl, naphthyl, tetrahydronaphthyl and indanyl.

“Arylalkyl” refers to an alkyl moiety substituted with an aryl group, wherein aryl is as defined herein.

“Carboxyalkylaminoalkyl” refers to a -(alkylenyl)-NH-(alkylenyl)-COOH group, of which one example is carboxymethylaminomethyl.

“Cycloalkyl” is a hydrocarbyl group containing at least one saturated or partially unsaturated ring structure, and attached via a ring carbon. In various embodiments, it refers to a saturated or a partially unsaturated C₃-C₁₂ cyclic moiety, examples of which include cyclopropyl, cyclobutyl, cyclopentyl, cyclopentenyl, cyclohexyl, cyclohexenyl, cycloheptyl and cyclooctyl.

“Cycloalkylalkyl” refers to an alkyl moiety substituted with a cycloalkyl group, wherein cycloalkyl is as defined herein. Examples of cycloalkylalkyl groups include cyclopropylmethyl, cyclobutylmethyl, cyclopentylethyl and cyclohexylmethyl.

“Dialkylamino” refers to an RR′N— group where R and R′ are independently alkyl as defined herein. Examples of dialkylamino groups include, but are not limited to, dimethylamino, diethylamino, methylethylamino and methylpropylamino. In various embodiments, R and R′ are independently a C₁-C₁₀ alkyl group or a C₁-C₆ alkyl group.

“Dialkylaminoalkyl” refers to an alkyl moiety substituted with a dialkylamino group, wherein dialkylamino is as defined herein. Examples of dialkylaminoalkyl groups include, but are not limited to, dimethylaminomethyl and diethylaminomethyl.

“Halo” refers to chloro (—Cl), bromo (—Br), fluoro (—F) or iodo (—I).

“Haloalkoxy” refers to an alkoxy group substituted with one or more halo groups and examples of haloalkoxy groups include, but are not limited to, —OCF₃, —OCHF₂ and —OCH₂F.

“Haloalkoxyalkyl” refers to an alkyl moiety substituted with a haloalkoxy group, wherein haloalkoxy is as defined herein. Examples of haloalkoxyalkyl groups include, but are not limited to, trifluoromethoxymethyl, trifluoroethoxymethyl and trifluoromethoxyethyl.

“Haloalkyl” refers to an alkyl moiety substituted with one or more halo groups. Examples of haloalkyl groups include —CF₃ and —CHF₂.

“Heteroaralkyl” refers to an alkyl moiety substituted with a heteroaryl group, wherein heteroaryl is as defined herein. Examples of heteroaralkyl groups include, but are not limited to, pyridinylmethyl and pyranylmethyl.

“Heteroaryl” refers to a monocyclic, bicyclic or tricyclic ring having up to 7 atoms in each ring, wherein at least one ring is aromatic and contains from 1 to 4 heteroatoms in the ring selected from the group consisting of N, O and S. Non-limiting examples of heteroaryl include pyridyl, thienyl, furanyl, pyrimidyl, imidazolyl, imidazopyridyl, pyranyl, pyrazolyl, pyrazolopyridyl, thiazolyl, thiadiazolyl, isothiazolyl, oxazolyl, isoxazoyl, pyrrolyl, pyridazinyl, pyrazinyl, quinolinyl, isoquinolinyl, benzofuranyl, dibenzofuranyl, dibenzothiophenyl, benzothienyl, indolyl, benzothiazolyl, benzooxazolyl, benzimidazolyl, isoindolyl, benzotriazolyl, purinyl, thianaphthenyl and pyrazinyl. Attachment of heteroaryl can occur via an aromatic ring, or, if heteroaryl is bicyclic or tricyclic and one of the rings is not aromatic or contains no heteroatoms, through a non-aromatic ring or a ring containing no heteroatoms. “Heteroaryl” is also understood to include the N-oxide derivative of any nitrogen containing heteroaryl.

“Heterocycloalkyl” refers to a saturated or partially unsaturated monovalent cyclic group of 3 to 8 ring-carbon atoms and, in addition to ring-carbon atoms, 1 to 2 hetero groups selected from N, O, or S(O)ₙ, wherein n is an integer selected from 0 through 2, inclusive. Examples of heterocycloalkyl groups include, but are not limited to, pyrrolidinylmethyl and piperidinylmethyl.

“Heterocycloalkylalkyl” refers to an alkyl moiety substituted with a heterocycloalkyl group, wherein heterocycloalkyl is as defined herein. Examples of heterocycloalkylalkyl groups include, but are not limited to, piperazinylmethyl and morpholinylethyl.

“Hydroxyalkoxy” refers to an alkoxy group substituted with a hydroxyl group (—OH), wherein alkoxy is as defined herein. An example of hydroxyalkoxy is hydroxyethoxy.

“Hydroxyalkoxyalkyl” refers to -(alkylenyl)-O-(alkylenyl)-OH or an alkyl moiety substituted with a hydroxyalkoxy, wherein hydroxyalkoxy is as defined herein. An example of hydroxyalkoxyalkyl is hydroxyethoxymethyl.

“Hydroxyalkyl” refers to a linear or branched monovalent C₁-C₆ hydrocarbon group substituted with at least one hydroxy group and examples of hydroxyalkyl groups include, but are not limited to, hydroxymethyl, hydroxyethyl, hydroxypropyl and hydroxybutyl.

“Pharmaceutically-acceptable” means suitable for use in pharmaceutical preparations, generally considered as safe for such use, officially approved by a regulatory agency of a national or state government for such use, or being listed in the U.S. Pharmacopoeia or other generally recognized pharmacopoeia for use in animals, and more particularly in humans.

“Pharmaceutically-acceptable carrier” refers to a diluent, adjuvant, excipient, or carrier, or other ingredient which is pharmaceutically-acceptable and with which a compound of this disclosure is administered.

“Pharmaceutically-acceptable salt” refers to a salt which may enhance desired pharmacological activity. Examples of pharmaceutically-acceptable salts include acid addition salts formed with inorganic or organic acids, metal salts and amine salts. Examples of acid addition salts formed with inorganic acids include salts with hydrochloric acid, hydrobromic acid, sulfuric acid, nitric acid and phosphoric acid. Examples of acid addition salts formed with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxy-benzoyl)-benzoic acid, cinnamic acid, mandelic acid, methanesulfonic acid, ethanesulfonic acid, 1,2-ethanedisulfonic acid, 2-hydroxyethane-sulfonic acid, benzenesulfonic acid, p-chlorobenzenesulfonic acid, 2-naphthalenesulfonic acid, p-toluenesulfonic acid, camphorsulfonic acid, 4-methyl-bicyclo[2.2.2]oct-2-ene1-carboxylic acid, gluco-heptonic acid, 4,4′-methylenebis(3-hydroxy-2-naphthoic) acid, 3-phenylpropionic acid, trimethyl-acetic acid, tertiary butylacetic acid, lauryl sulfuric acid, gluconic acid, glutamic acid, hydroxy-naphthoic acids, salicylic acid, stearic acid and muconic acid. Examples of metal salts include salts with sodium, potassium, calcium, magnesium, aluminum, iron, and zinc ions. Examples of amine salts include salts with ammonia and organic nitrogenous bases strong enough to form salts with carboxylic acids.

“Therapeutically-effective amount” refers to an amount of a compound that, when administered to a subject for treating a disease, is sufficient to effect treatment for the disease. “Therapeutically effective amount” can vary depending on the compound, the disease and its severity, the age, the weight, etc. of the subject to be treated.

Embraced herein, where applicable, are permissible isomers such as tautomers, racemates, enantiomers, diastereomers, atropisomers, configurational isomers of double bonds (E- and/or Z-), cis- and trans-configurations in ring substitution patterns, and isotopic variants.

In one embodiment, there is provided a compound of Formula (I) or a pharmaceutically acceptable salt thereof:

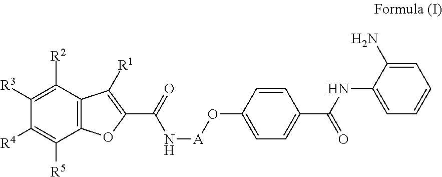

wherein R¹ is selected from the group consisting of H, alkyl, cycloalkyl, alkoxy, amino, aminoalkyl and aryl; and if R¹ is not H, R¹ is optionally substituted with halo, hydroxyl, alkyl, cycloalkyl, alkoxy, amino, aryl, haloaryl, heteroaryl or heterocycloalkyl;

R², R³, R⁴ and R⁵ are independently selected from the group consisting of H, alkyl, cycloalkyl, alkoxy, amino, aminoalkyl, aminoalkoxy, carboxyalkylaminoalkyl and aryl; and if any of R², R³, R⁴ and R⁵ is not H, each of R², R³, R⁴ and R⁵ is optionally substituted with halo, hydroxyl, alkyl, cycloalkyl, alkoxy, amino, aryl, haloaryl, heteroaryl or heterocycloalkyl; and

A is C₂-C₆ alkylenyl optionally substituted with hydroxyl or alkyl, or C₄-C₆ alkenylenyl optionally substituted with alkyl.

The compound defined above is useful to inhibit histone deacetylases. In one embodiment, therefore, a compound of the disclosure is used in inhibiting HDAC enzymes such as, for example, mammalian HDAC enzymes. More specifically, a compound of this disclosure may inhibit class I HDAC enzymes selectively over class II HDAC and thus can be used to treat diseases in which the major pathological factor is class I HDAC. Most specifically, a compound of the disclosure can be used to treat or inhibit HDAC 1-mediated diseases or abnormalities.

In one embodiment, the disclosure provides a compound of Formula (I) having at least one aryl-containing-substituent, wherein aryl is phenyl; haloaryl is mono-fluorophenyl such as 2-, 3- or 4-fluorophenyl; and haloalkylaryl is mono-trifluoromethylphenyl such as 2-, 3- or 4-trifluoromethylphenyl.

In another embodiment, the present disclosure provides a compound of Formula (I) having at least one heteroaryl-containing-substituent, wherein heteroaryl is selected from the group consisting of pyridinyl, pyranyl and imidazolyl.

In yet another embodiment, the disclosure provides a compound of Formula (I) having at least one heterocycloalkyl-containing substituent, wherein heterocycloalkyl is selected from the group consisting of pyrrolidinyl, piperidinyl, thiazinyl and morpholinyl.

In yet another embodiment, the disclosure provides a compound of Formula (I) wherein at least one of R¹, R², R³, R⁴ and R⁵ has a halo optional substitution, and the halo is fluoro. Therefore, R¹, R², R³, R⁴ and R⁵ optionally substituted with halo group can be, for example, fluoroalkyl, fluoroalkoxy and fluoroaryl.

In yet another embodiment, the disclosure provides a compound of Formula (I) wherein R¹ is selected from the group consisting of methyl, ethyl, propyl, methoxy, ethoxy, methoxymethyl, ethoxyethyl, propoxyethyl, methoxyethoxy, trifluoromethyl, hydroxyethoxy, dimethylamino, diethylamino, dimethylaminomethyl, diethylaminomethyl, dimethylaminoethoxy, trifluoromethoxymethyl, trifluoroethoxymethyl, benzyl, phenylethyl, trifluoromethylphenylethyl, phenoxymethyl, fluorophenoxymethyl, phenylethylaminomethyl, benzylaminomethyl, morpholinylmethyl, morpholinylethoxy, imidazolylmethyl, triazinylmethyl, piperidinylmethyl, trifluoromethylpiperidinylmethyl, pyridinylmethoxy, methylpiperazinylmethyl, pyrrolidinylmethyl and pyrrolidinylethoxy;

R², R³, R⁴ and R⁵ are independently selected from the group consisting of methyl, ethyl, propyl, methoxy, ethoxy, methoxymethyl, ethoxyethyl, propoxyethyl, methoxyethoxy, trifluoromethyl, hydroxyethoxy, dimethylamino, diethylamino, dimethylaminomethyl, diethylaminomethyl, dimethylaminoethoxy, trifluoromethoxymethyl, trifluoroethoxymethyl, benzyl, phenylethyl, trifluoromethylphenylethyl, phenoxymethyl, fluorophenoxymethyl, phenylethylaminomethyl, benzylaminomethyl, morpholinylmethyl, morpholinylethoxy, imidazolylmethyl, triazinylmethyl, piperidinylmethyl, trifluoromethylpiperidinylmethyl, pyridinylmethoxy, methylpiperazinylmethyl, pyrrolidinylmethyl and pyrrolidinylethoxy; and

A may be an ethylenyl or propylenyl linker, each optionally substituted with methyl or ethyl.

In some embodiments, only one of R¹, R², R³, R⁴ and R⁵ is non-hydrogen and A is a saturated divalent aliphatic linker.

In an embodiment, the present disclosure provides a compound having the formula

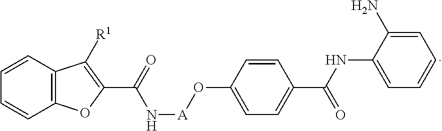

Examples of compounds having the structure above wherein A is ethylenyl or propylenyl include

N-(2-aminophenyl)-4-[2-(benzofuran-2-ylcarbonylamino)-ethoxy]-benzamide;
N-(2-aminophenyl)-4-[3-(benzofuran-2-ylcarbonylamino)-propoxy]-benzamide;
N-(2-aminophenyl)-4-[2-(3-methyl benzofuran-2-ylcarbonylamino)-ethoxy]-benzamide;
N-(2-aminophenyl)-4-[3-(3-methyl benzofuran-2-yl carbonylamino)-propoxy]-benzamide;
N-(2-aminophenyl)-4-[2-(3-ethyl benzofuran-2-ylcarbonylamino)-ethoxy]-benzamide;
N-(2-aminophenyl)-4-[2-(3-propyl benzofuran-2-ylcarbonylamino)-ethoxy]-benzamide;
N-(2-aminophenyl)-4-[2-(3-methoxy benzofuran-2-ylcarbonylamino)-ethoxy]-benzamide;
N-(2-aminophenyl)-4-[3-(3-methoxy benzofuran-2-ylcarbonylamino)-propoxy]-benzamide;
N-(2-aminophenyl)-4-[2-(3-ethoxy benzofuran-2-ylcarbonylamino)-ethoxy]-benzamide;
N-(2-aminophenyl)-4-[2-(3-methoxymethyl benzofuran-2-ylcarbonylamino)-ethoxy]-benzamide;
N-(2-aminophenyl)-4-[3-(3-methoxymethyl benzofuran-2-ylcarbonylamino)-propoxy]-benzamide;
N-(2-aminophenyl)-4-[2-(3-ethoxyethyl benzofuran-2-yl carbonylamino)-ethoxy]-benzamide;
N-(2-aminophenyl)-4-[2-(3-propoxyethyl benzofuran-2-yl carbonylamino)-ethoxy]-benzamide;
N-(2-aminophenyl)-4-[2-(3-methoxyethoxy benzofuran-2-yl carbonylamino)-ethoxy]-benzamide;
N-(2-aminophenyl)-4-[3-(3-methoxyethoxy benzofuran-2-yl carbonylamino)-propoxy]-benzamide;
N-(2-aminophenyl)-4-[2-(3-trifluoromethyl benzofuran-2-yl carbonylamino)-ethoxy]-benzamide;
N-(2-aminophenyl)-4-[2-(3-hydroxyethoxy benzofuran-2-yl carbonylamino)-ethoxy]-benzamide;
N-(2-aminophenyl)-4-[2-(3-dimethylamino benzofuran-2-yl carbonylamino)-ethoxy]-benzamide;
N-(2-aminophenyl)-4-[2-(3-diethylamino benzofuran-2-yl carbonylamino)-ethoxy]-benzamide;
N-(2-aminophenyl)-4-[2-(3-dimethylaminomethyl benzofuran-2-yl carbonylamino)-ethoxy]-benzamide;
N-(2-aminophenyl)-4-[2-(3-diethylaminomethyl benzofuran-2-yl carbonylamino)-ethoxy]-benzamide;
N-(2-aminophenyl)-4-[2-(3-dimethylaminoethoxy benzofuran-2-yl carbonylamino)-ethoxy]-benzamide;
N-(2-aminophenyl)-4-[2-(3-trifluoromethoxymethyl benzofuran-2-yl carbonylamino)-ethoxy]-benzamide;
N-(2-aminophenyl)-4-[2-(3-trifluoroethoxymethyl benzofuran-2-yl carbonylamino)-ethoxy]-benzamide;
N-(2-aminophenyl)-4-[2-(3-phenylethyl benzofuran-2-yl carbonylamino)-ethoxy]-benzamide;
N-(2-aminophenyl)-4-[2-(3-trifluoromethylphenylethyl benzofuran-2-yl carbonylamino)-ethoxy]-benzamide;
N-(2-aminophenyl)-4-[2-(3-phenoxymethyl benzofuran-2-yl carbonylamino)-ethoxy]benzamide;
N-(2-aminophenyl)-4-[3-(3-phenoxymethyl benzofuran-2-yl carbonylamino)-propoxy]-benzamide;
N-(2-aminophenyl)-4-[2-(3-fluorophenoxymethyl benzofuran-2-yl carbonylamino)-ethoxy]-benzamide;
N-(2-aminophenyl)-4-[2-(3-phenylethylaminomethyl benzofuran-2-yl carbonylamino)-ethoxy]-benzamide;
N-(2-aminophenyl)-4-[2-(3-benzylaminomethyl benzofuran-2-yl carbonylamino)-ethoxy]-benzamide;
N-(2-aminophenyl)-4-[2-(3-morpholinylmethyl benzofuran-2-yl carbonylamino)-ethoxy]-benzamide;
N-(2-aminophenyl)-4-[2-(3-morpholinylethoxy benzofuran-2-yl carbonylamino)-ethoxy]-benzamide;
N-(2-aminophenyl)-4-[2-(3-imidazolylmethyl benzofuran-2-yl carbonylamino)-ethoxy]-benzamide;
N-(2-aminophenyl)-4-[2-(3-triazinylmethyl benzofuran-2-yl carbonylamino)-ethoxy]-benzamide;
N-(2-aminophenyl)-4-[2-(3-piperidinylmethyl benzofuran-2-yl carbonylamino)-ethoxy]-benzamide;
N-(2-aminophenyl)-4-[2-(3-trifluoromethylpiperidinylmethyl benzofuran-2-yl carbonylamino)-ethoxy]-benzamide;
N-(2-aminophenyl)-4-[2-(3-pyridinylmethoxy benzofuran-2-yl carbonylamino)-ethoxy]-benzamide;
N-(2-aminophenyl)-4-[2-(3-methylpiperazinylmethyl benzofuran-2-yl carbonylamino)-ethoxy]-benzamide;
N-(2-aminophenyl)-4-[2-(3-pyrrolidinylmethyl benzofuran-2-yl carbonylamino)-ethoxy]-benzamide; and
N-(2-aminophenyl)-4-[2-(3-pyrrolidinylethoxy benzofuran-2-yl carbonylamino)-ethoxy]-benzamide.

In an embodiment, the disclosure provides a compound wherein A is ethylenyl or propylenyl having the formula

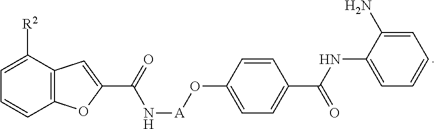

Examples of compounds having the structure above wherein A is ethylenyl or propylenyl include

N-(2-aminophenyl)-4-[2-(4-methyl benzofuran-2-yl carbonylamino)-ethoxy]-benzamide;
N-(2-aminophenyl)-4-[3-(4-methyl benzofuran-2-yl carbonylamino)-propoxy]-benzamide;
N-(2-aminophenyl)-4-[2-(4-methyl benzofuran-2-yl carbonylamino)-ethoxy]-benzamide
N-(2-aminophenyl)-4-[2-(4-propyl benzofuran-2-yl carbonylamino)-ethoxy]-benzamide;
N-(2-aminophenyl)-4-[2-(4-methoxy benzofuran-2-yl carbonylamino)-ethoxy]-benzamide;
N-(2-aminophenyl)-4-[3-(4-methoxy benzofuran-2-yl carbonylamino)-propoxy]-benzamide;
N-(2-aminophenyl)-4-[2-(4-ethoxy benzofuran-2-yl carbonylamino)-ethoxy]-benzamide;
N-(2-aminophenyl)-4-[2-(4-methoxymethyl benzofuran-2-yl carbonylamino)-ethoxy]-benzamide;
N-(2-aminophenyl)-4-[3-(4-methoxymethyl benzofuran-2-yl carbonylamino)-propoxy]-benzamide;
N-(2-aminophenyl)-4-[2-(4-ethoxyethyl benzofuran-2-yl carbonylamino)-ethoxy]-benzamide;
N-(2-aminophenyl)-4-[2-(4-propoxyethyl benzofuran-2-yl carbonylamino)-ethoxy]-benzamide;
N-(2-aminophenyl)-4-[2-(4-methoxyethoxy benzofuran-2-yl carbonylamino)-ethoxy]-benzamide;
N-(2-aminophenyl)-4-[3-(4-methoxyethoxy benzofuran-2-yl carbonylamino)-propoxy]-benzamide;
N-(2-aminophenyl)-4-[2-(4-trifluoromethyl benzofuran-2-yl carbonylamino)-ethoxy]-benzamide;
N-(2-aminophenyl)-4-[2-(4-hydroxyethoxy benzofuran-2-yl carbonylamino)-ethoxy]-benzamide;
N-(2-aminophenyl)-4-[2-(4-dimethylamino benzofuran-2-yl carbonylamino)-ethoxy]-benzamide;
N-(2-aminophenyl)-4-[2-(4-diethylamino benzofuran-2-yl carbonylamino)-ethoxy]-benzamide;
N-(2-aminophenyl)-4-[2-(4-dimethylaminomethyl benzofuran-2-yl carbonylamino)-ethoxy]-benzamide;
N-(2-aminophenyl)-4-[2-(4-diethylaminomethyl benzofuran-2-yl carbonylamino)-ethoxy]-benzamide;
N-(2-aminophenyl)-4-[2-(4-dimethylaminoethoxy benzofuran-2-yl carbonylamino)-ethoxy]-benzamide;
N-(2-aminophenyl)-4-[2-(4-trifluoromethoxymethyl benzofuran-2-yl carbonylamino)-ethoxy]-benzamide;
N-(2-aminophenyl)-4-[2-(4-trifluoroethoxymethyl benzofuran-2-yl carbonylamino)-ethoxy]-benzamide;
N-(2-aminophenyl)-4-[2-(4-phenylethyl benzofuran-2-yl carbonylamino)-ethoxy]-benzamide;
N-(2-aminophenyl)-4-[2-(4-trifluoromethylphenylethyl benzofuran-2-yl carbonylamino)-ethoxy]-benzamide;
N-(2-aminophenyl)-4-[2-(4-phenoxymethyl benzofuran-2-yl carbonylamino)-ethoxy]-benzamide;
N-(2-aminophenyl)-4-[3-(4-phenoxymethyl benzofuran-2-yl carbonylamino)-propoxy]-benzamide;
N-(2-aminophenyl)-4-[2-(4-fluorophenoxymethyl benzofuran-2-yl carbonylamino)-ethoxy]-benzamide;
N-(2-aminophenyl)-4-[2-(4-phenylethylaminomethyl benzofuran-2-yl carbonylamino)-ethoxy]-benzamide;
N-(2-aminophenyl)-4-[2-(4-benzylaminomethyl benzofuran-2-yl carbonylamino)-ethoxy]-benzamide;
N-(2-aminophenyl)-4-[2-(4-morpholinylmethyl benzofuran-2-yl carbonylamino)-ethoxy]-benzamide;
N-(2-aminophenyl)-4-[2-(4-morpholinylethoxy benzofuran-2-yl carbonylamino)-ethoxy]-benzamide;
N-(2-aminophenyl)-4-[2-(4-imidazolylmethyl benzofuran-2-yl carbonylamino)-ethoxy]-benzamide;
N-(2-aminophenyl)-4-[2-(4-triazinylmethyl benzofuran-2-yl carbonylamino)-ethoxy]-benzamide;
N-(2-aminophenyl)-4-[2-(4-piperidinylmethyl benzofuran-2-yl carbonylamino)-ethoxy]-benzamide;
N-(2-aminophenyl)-4-[2-(4-trifluoromethylpiperidinylmethyl benzofuran-2-yl carbonylamino)-ethoxy]-benzamide;
N-(2-aminophenyl)-4-[2-(4-pyridinylmethoxy benzofuran-2-yl carbonylamino)-ethoxy]-benzamide;
N-(2-aminophenyl)-4-[2-(4-methylpiperazinylmethyl benzofuran-2-yl carbonylamino)-ethoxy]-benzamide;
N-(2-aminophenyl)-4-[2-(4-pyrrolidinylmethyl benzofuran-2-yl carbonylamino)-ethoxy]-benzamide; and
N-(2-aminophenyl)-4-[2-(4-pyrrolidinylethoxy benzofuran-2-yl carbonylamino)-ethoxy]-benzamide.

In an embodiment, the present disclosure provides a compound having the formula

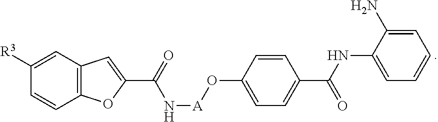

Examples of compounds having the structure above wherein A is ethylenyl or propylenyl include

N-(2-aminophenyl)-4-[2-(5-methyl benzofuran-2-yl carbonylamino)-ethoxy]-benzamide;
N-(2-aminophenyl)-4-[3-(5-methyl benzofuran-2-yl carbonylamino)-propoxy]-benzamide;
N-(2-aminophenyl)-4-[2-(5-ethyl benzofuran-2-yl carbonylamino)-ethoxy]-benzamide
N-(2-aminophenyl)-4-[2-(5-propyl benzofuran-2-yl carbonylamino)-ethoxy]-benzamide;
N-(2-aminophenyl)-4-[2-(5-methoxy benzofuran-2-yl carbonylamino)-ethoxy]-benzamide;
N-(2-aminophenyl)-4-[3-(5-methoxy benzofuran-2-yl carbonylamino)-propoxy]-benzamide;
N-(2-aminophenyl)-4-[2-(5-ethoxy benzofuran-2-yl carbonylamino)-ethoxy]-benzamide;
N-(2-aminophenyl)-4-[2-(5-methoxymethyl benzofuran-2-yl carbonylamino)-ethoxy]-benzamide;
N-(2-aminophenyl)-4-[3-(5-methoxymethyl benzofuran-2-yl carbonylamino)-propoxy]-benzamide;
N-(2-aminophenyl)-4-[2-(5-ethoxyethyl benzofuran-2-yl carbonylamino)-ethoxy]-benzamide;
N-(2-aminophenyl)-4-[2-(5-propoxyethyl benzofuran-2-yl carbonylamino)-ethoxy]-benzamide;
N-(2-aminophenyl)-4-[2-(5-methoxyethoxy benzofuran-2-yl carbonylamino)-ethoxy]-benzamide;
N-(2-aminophenyl)-4-[2-(5-methoxyethoxy benzofuran-2-yl carbonylamino)-ethoxy]-benzamide;
N-(2-aminophenyl)-4-[3-(5-trifluoromethyl benzofuran-2-yl carbonylamino)-propoxy]-benzamide;
N-(2-aminophenyl)-4-[2-(5-hydroxyethoxy benzofuran-2-yl carbonylamino)-ethoxy]-benzamide;
N-(2-aminophenyl)-4-[2-(5-dimethylamino benzofuran-2-yl carbonylamino)-ethoxy]-benzamide;
N-(2-aminophenyl)-4-[2-(5-diethylamino benzofuran-2-yl carbonylamino)-ethoxy]-benzamide;
N-(2-aminophenyl)-4-[2-(5-dimethylaminomethyl benzofuran-2-yl carbonylamino)-ethoxy]-benzamide;
N-(2-aminophenyl)-4-[2-(5-diethylaminomethyl benzofuran-2-yl carbonylamino)-ethoxy]-benzamide;
N-(2-aminophenyl)-4-[2-(5-dimethylaminoethoxy benzofuran-2-yl carbonylamino)-ethoxy]-benzamide;
N-(2-aminophenyl)-4-[2-(5-trifluoromethoxymethyl benzofuran-2-yl carbonylamino)-ethoxy]-benzamide;
N-(2-aminophenyl)-4-[2-(5-trifluoroethoxymethyl benzofuran-2-yl carbonylamino)-ethoxy]-benzamide;
N-(2-aminophenyl)-4-[2-(5-phenylethyl benzofuran-2-yl carbonylamino)-ethoxy]-benzamide;
N-(2-aminophenyl)-4-[2-(5-trifluoromethylphenylethyl benzofuran-2-yl carbonylamino)-ethoxy]-benzamide;
N-(2-aminophenyl)-4-[2-(5-phenoxymethyl benzofuran-2-yl carbonylamino)-ethoxy]-benzamide;
N-(2-aminophenyl)-4-[3-(5-phenoxymethyl benzofuran-2-yl carbonylamino)-propoxy]-benzamide;
N-(2-aminophenyl)-4-[2-(5-fluorophenoxymethyl benzofuran-2-yl carbonylamino)-ethoxy]-benzamide;
N-(2-aminophenyl)-4-[2-(5-phenylethylaminomethyl benzofuran-2-yl carbonylamino)-ethoxy]-benzamide;
N-(2-aminophenyl)-4-[2-(5-benzylaminomethyl benzofuran-2-yl carbonylamino)-ethoxy]-benzamide;
N-(2-aminophenyl)-4-[2-(5-morpholinylmethyl benzofuran-2-yl carbonylamino)-ethoxy]-benzamide;
N-(2-aminophenyl)-4-[2-(5-morpholinylethoxy benzofuran-2-yl carbonylamino)-ethoxy]-benzamide;
N-(2-aminophenyl)-4-[2-(5-imidazolylmethyl benzofuran-2-yl carbonylamino)-ethoxy]-benzamide;
N-(2-aminophenyl)-4-[2-(5-triazinylmethyl benzofuran-2-yl carbonylamino)-ethoxy]-benzamide;
N-(2-aminophenyl)-4-[2-(5-piperidinylmethyl benzofuran-2-yl carbonylamino)-ethoxy]-benzamide;
N-(2-aminophenyl)-4-[2-(5-trifluoromethylpiperidinylmethyl benzofuran-2-yl carbonylamino)-ethoxy]-benzamide;
N-(2-aminophenyl)-4-[2-(5-pyridinylmethoxy benzofuran-2-yl carbonylamino)-ethoxy]-benzamide;
N-(2-aminophenyl)-4-[2-(5-methylpiperazinylmethyl benzofuran-2-yl carbonylamino)-ethoxy]-benzamide;
N-(2-aminophenyl)-4-[2-(5-pyrrolidinylmethyl benzofuran-2-yl carbonylamino)-ethoxy]-benzamide; and
N-(2-aminophenyl)-4-[2-(5-pyrrolidinylethoxy benzofuran-2-yl carbonylamino)-ethoxy]-benzamide.

In an embodiment, the present disclosure provides a compound having the formula

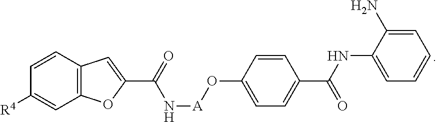

Examples of compounds having the structure above wherein A is ethylenyl or propylenyl include N-(2-aminophenyl)-4-[2-(6-methyl benzofuran-2-yl carbonylamino)-ethoxy]-benzamide;

N-(2-aminophenyl)-4-[3-(6-methyl benzofuran-2-yl carbonylamino)-propoxy]-benzamide;
N-(2-aminophenyl)-4-[2-(6-ethyl benzofuran-2-yl carbonylamino)-ethoxy]-benzamide
N-(2-aminophenyl)-4-[2-(6-propyl benzofuran-2-yl carbonylamino)-ethoxy]-benzamide;
N-(2-aminophenyl)-4-[2-(6-methoxy benzofuran-2-yl carbonylamino)-ethoxy]-benzamide;
N-(2-aminophenyl)-4-[3-(6-methoxy benzofuran-2-yl carbonylamino)-propoxy]-benzamide;
N-(2-aminophenyl)-4-[2-(6-ethoxy benzofuran-2-yl carbonylamino)-ethoxy]-benzamide;
N-(2-aminophenyl)-4-[2-(6-methoxymethyl benzofuran-2-yl carbonylamino)-ethoxy]-benzamide;
N-(2-aminophenyl)-4-[3-(6-methoxymethyl benzofuran-2-yl carbonylamino)-propoxy]-benzamide;
N-(2-aminophenyl)-4-[2-(6-ethoxyethyl benzofuran-2-yl carbonylamino)-ethoxy]-benzamide;
N-(2-aminophenyl)-4-[2-(6-propoxyethyl benzofuran-2-yl carbonylamino)-ethoxy]-benzamide;
N-(2-aminophenyl)-4-[2-(6-methoxyethoxy benzofuran-2-yl carbonylamino)-ethoxy]-benzamide;
N-(2-aminophenyl)-4-[3-(6-methoxyethoxy benzofuran-2-yl carbonylamino)-propoxy]-benzamide;
N-(2-aminophenyl)-4-[2-(6-trifluoromethyl benzofuran-2-yl carbonylamino)-ethoxy]-benzamide;
N-(2-aminophenyl)-4-[2-(6-hydroxyethoxy benzofuran-2-yl carbonylamino)-ethoxy]-benzamide;
N-(2-aminophenyl)-4-[2-(6-dimethylamino benzofuran-2-yl carbonylamino)-ethoxy]-benzamide;
N-(2-aminophenyl)-4-[2-(6-diethylamino benzofuran-2-yl carbonylamino)-ethoxy]-benzamide;
N-(2-aminophenyl)-4-[2-(6-dimethylaminomethyl benzofuran-2-yl carbonylamino)-ethoxy]-benzamide;
N-(2-aminophenyl)-4-[2-(6-diethylaminomethyl benzofuran-2-yl carbonylamino)-ethoxy]-benzamide;
N-(2-aminophenyl)-4-[2-(6-dimethylaminoethoxy benzofuran-2-yl carbonylamino)-ethoxy]-benzamide;
N-(2-aminophenyl)-4-[2-(6-trifluoromethoxymethyl benzofuran-2-yl carbonylamino)-ethoxy]-benzamide;
N-(2-aminophenyl)-4-[2-(6-trifluoroethoxymethyl benzofuran-2-yl carbonylamino)-ethoxy]-benzamide;
N-(2-aminophenyl)-4-[2-(6-phenylethyl benzofuran-2-yl carbonylamino)-ethoxy]-benzamide;
N-(2-aminophenyl)-4-[2-(6-trifluoromethylphenylethyl benzofuran-2-yl carbonylamino)-ethoxy]-benzamide;
N-(2-aminophenyl)-4-[2-(6-phenoxymethyl benzofuran-2-yl carbonylamino)-ethoxy]-benzamide;
N-(2-aminophenyl)-4-[3-(6-phenoxymethyl benzofuran-2-yl carbonylamino)-propoxy]-benzamide;
N-(2-aminophenyl)-4-[2-(6-fluorophenoxymethyl benzofuran-2-yl carbonylamino)-ethoxy]-benzamide;
N-(2-aminophenyl)-4-[2-(6-phenylethylaminomethyl benzofuran-2-yl carbonylamino)-ethoxy]-benzamide;
N-(2-aminophenyl)-4-[2-(6-benzylaminomethyl benzofuran-2-yl carbonylamino)-ethoxy]-benzamide;
N-(2-aminophenyl)-4-[2-(6-morpholinylmethyl benzofuran-2-yl carbonylamino)-ethoxy]-benzamide;
N-(2-aminophenyl)-4-[2-(6-morpholinylethoxy benzofuran-2-yl carbonylamino)-ethoxy]-benzamide;
N-(2-aminophenyl)-4-[2-(6-imidazolylmethyl benzofuran-2-yl carbonylamino)-ethoxy]-benzamide;
N-(2-aminophenyl)-4-[2-(6-triazinylmethyl benzofuran-2-yl carbonylamino)-ethoxy]-benzamide;
N-(2-aminophenyl)-4-[2-(6-piperidinylmethyl benzofuran-2-yl carbonylamino)-ethoxy]-benzamide;
N-(2-aminophenyl)-4-[2-(6-trifluoromethylpiperidinylmethyl benzofuran-2-yl carbonylamino)-ethoxy]-benzamide;
N-(2-aminophenyl)-4-[2-(6-pyridinylmethoxy benzofuran-2-yl carbonylamino)-ethoxy]-benzamide;
N-(2-aminophenyl)-4-[2-(6-methylpiperazinylmethyl benzofuran-2-yl carbonylamino)-ethoxy]-benzamide;
N-(2-aminophenyl)-4-[2-(6-pyrrolidinylmethyl benzofuran-2-yl carbonylamino)-ethoxy]benzamide; and
N-(2-aminophenyl)-4-[2-(6-pyrrolidinylethoxy benzofuran-2-yl carbonylamino)-ethoxy]-benzamide.

In an embodiment, the present disclosure provides a compound having the formula,

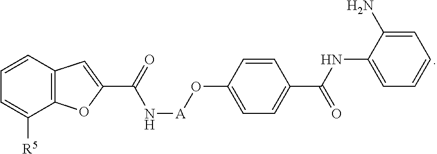

Examples of compounds having the structure above wherein A is ethylenyl or propylenyl include

N-(2-aminophenyl)-4-[2-(7-methyl benzofuran-2-yl carbonylamino)-ethoxy]-benzamide;
N-(2-aminophenyl)-4-[3-(7-methyl benzofuran-2-yl carbonylamino)-propoxy]-benzamide;
N-(2-aminophenyl)-4-[2-(7-ethyl benzofuran-2-yl carbonylamino)-ethoxy]-benzamide
N-(2-aminophenyl)-4-[2-(7-propyl benzofuran-2-yl carbonylamino)-ethoxy]-benzamide;
N-(2-aminophenyl)-4-[2-(7-methoxy benzofuran-2-yl carbonylamino)-ethoxy]-benzamide;
N-(2-aminophenyl)-4-[3-(7-methoxy benzofuran-2-yl carbonylamino)-propoxy]-benzamide;
N-(2-aminophenyl)-4-[2-(7-ethoxy benzofuran-2-yl carbonylamino)-ethoxy]-benzamide;
N-(2-aminophenyl)-4-[2-(7-methoxymethyl benzofuran-2-yl carbonylamino)-ethoxy]-benzamide;
N-(2-aminophenyl)-4-[3-(7-methoxymethyl benzofuran-2-yl carbonylamino)-propoxy]-benzamide;
N-(2-aminophenyl)-4-[2-(7-ethoxyethyl benzofuran-2-yl carbonylamino)-ethoxy]-benzamide;
N-(2-aminophenyl)-4-[2-(7-propoxyethyl benzofuran-2-yl carbonylamino)-ethoxy]-benzamide;
N-(2-aminophenyl)-4-[2-(7-methoxyethoxy benzofuran-2-yl carbonylamino)-ethoxy]-benzamide;
N-(2-aminophenyl)-4-[3-(7-methoxyethoxy benzofuran-2-yl carbonylamino)-propoxy]-benzamide;
N-(2-aminophenyl)-4-[2-(7-trifluoromethyl benzofuran-2-yl carbonylamino)-ethoxy]-benzamide;
N-(2-aminophenyl)-4-[2-(7-hydroxyethoxy benzofuran-2-yl carbonylamino)-ethoxy]-benzamide;
N-(2-aminophenyl)-4-[2-(7-dimethylamino benzofuran-2-yl carbonylamino)-ethoxy]-benzamide;
N-(2-aminophenyl)-4-[2-(7-diethylamino benzofuran-2-yl carbonylamino)-ethoxy]-benzamide;
N-(2-aminophenyl)-4-[2-(7-dimethylaminomethyl benzofuran-2-yl carbonylamino)-ethoxy]-benzamide;
N-(2-aminophenyl)-4-[2-(7-diethylaminomethyl benzofuran-2-yl carbonylamino)-ethoxy]-benzamide;
N-(2-aminophenyl)-4-[2-(7-dimethylaminoethoxy benzofuran-2-yl carbonylamino)-ethoxy]-benzamide;
N-(2-aminophenyl)-4-[2-(7-trifluoromethoxymethyl benzofuran-2-yl carbonylamino)-ethoxy]-benzamide;
N-(2-aminophenyl)-4-[2-(7-trifluoroethoxymethyl benzofuran-2-yl carbonylamino)-ethoxy]-benzamide;
N-(2-aminophenyl)-4-[2-(7-phenylethyl benzofuran-2-yl carbonylamino)-ethoxy]-benzamide;
N-(2-aminophenyl)-4-[2-(7-trifluoromethylphenylethyl benzofuran-2-yl carbonylamino)-ethoxy]-benzamide;
N-(2-aminophenyl)-4-[2-(7-phenoxymethyl benzofuran-2-yl carbonylamino)-ethoxy]-benzamide;
N-(2-aminophenyl)-4-[2-(7-fluorophenoxymethyl benzofuran-2-yl carbonylamino)-ethoxy]-benzamide;
N-(2-aminophenyl)-4-[2-(7-phenylethylaminomethyl benzofuran-2-yl carbonylamino)-ethoxy]-benzamide;
N-(2-aminophenyl)-4-[2-(7-benzylaminomethyl benzofuran-2-yl carbonylamino)-ethoxy]-benzamide;
N-(2-aminophenyl)-4-[2-(7-morpholinylmethyl benzofuran-2-ylcarbonylamino)-ethoxy]-benzamide;
N-(2-aminophenyl)-4-[2-(7-morpholinylethoxy benzofuran-2-yl carbonylamino)-ethoxy]-benzamide;
N-(2-aminophenyl)-4-[2-(7-imidazolylmethyl benzofuran-2-ylcarbonylamino)-ethoxy]-benzamide;
N-(2-aminophenyl)-4-[2-(7-triazinylmethyl benzofuran-2-yl carbonylamino)-ethoxy]-benzamide;
N-(2-aminophenyl)-4-[2-(7-piperidinylmethyl benzofuran-2-yl carbonylamino)-ethoxy]-benzamide;
N-(2-aminophenyl)-4-[2-(7-trifluoromethylpiperidinylmethyl benzofuran-2-yl carbonylamino)-ethoxy]-benzamide;
N-(2-aminophenyl)-4-[2-(7-pyridinylmethoxy benzofuran-2-yl carbonylamino)-ethoxy]-benzamide;
N-(2-aminophenyl)-4-[2-(7-methylpiperazinylmethyl benzofuran-2-yl carbonylamino)-ethoxy]-benzamide;
N-(2-aminophenyl)-4-[2-(7-pyrrolidinylmethyl benzofuran-2-ylcarbonylamino)-ethoxy]-benzamide; and
N-(2-aminophenyl)-4-[2-(7-pyrrolidinylethoxy benzofuran-2-yl carbonylamino)-ethoxy]-benzamide.

In an embodiment, a compound with a benzofuran substituent of the present disclosure is

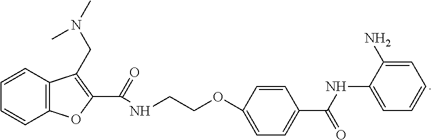

Compound Preparation

A compound of the present disclosure can be prepared according to the schemes described below, but it shall be appreciated that modifications of the illustrated process or other process can also be used. General Scheme A illustrates a method to prepare a compound of Formula (I).

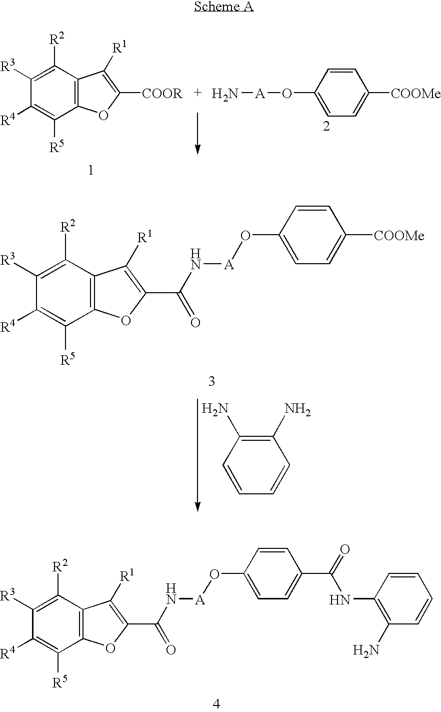

wherein R¹, R², R³, R⁴ and R⁵ are as defined herein and A is alkylenyl or C₄-C₆ alkenylenyl not having a double bond at a carbon atom adjacent to the amino group.

A catalyst promoting a condensation reaction is added to starting materials 1 and 2 in an organic solvent to produce intermediate 3. A coupling agent is added to a solution of intermediate 3 in an organic solvent. Benzene-1,2-diamine is reacted with the solution to produce compound 4.

The compounds of the present disclosure inhibit histone deacetylase activity and are useful to treat or ameliorate diseases mediated directly or indirectly by HDAC. Therefore, another aspect of the present disclosure is to provide a pharmaceutical composition comprising an effective amount of one or more compounds as described above.

In one embodiment of the disclosure, a pharmaceutical composition is provided comprising, in addition to one or more compounds described herein, at least one pharmaceutically-acceptable diluent, adjuvant, excipient, or carrier. The composition can take any suitable form for the desired route of administration. Where the composition is to be administered orally, any suitable orally deliverable dosage form can be used, including without limitation tablets, capsules (solid- or liquid-filled), powders, granules, syrups and other liquids, elixirs, inhalants, troches, lozenges, and solutions. Injectable compositions or iv infusions are also provided in the form of solutions, suspensions, and emulsions.

A pharmaceutical composition according to the present disclosure may contain one or more additional therapeutic agents, for example, to increase the efficacy or decrease the side effects. In some embodiments, accordingly, a pharmaceutical composition further contains one or more additional therapeutic agents selected from active ingredients useful to treat or inhibit diseases mediated directly or indirectly by HDAC. Examples of such active ingredients are, without limitation, agents to treat or inhibit cancer, Huntington's disease, cystic fibrosis, liver fibrosis, renal fibrosis, pulmonary fibrosis, skin fibrosis, Rheumatoid arthritis, diabetes, stroke, amyotrophic lateral sclerosis, cardiac hypertrophy, congestive heart failure, or Alzheimer's disease.

In an embodiment, an additional therapeutic agent to be included is an anti-cancer agent. Examples of an anti-cancer agent include, but are not limited to, alkylating agents such as cyclophosphamide, dacarbazine, and cisplatin; antimetabolites such as methotrexate, mercaptopurine, thioguanine, fluorouracil, and cytarabine; plant alkaloids such as vinblastine, and paclitaxel; antitumor antibiotics such as doxorubicin, bleomycin, and mitomycin; hormones/antihormones such as prednisone, tamoxifen, and flutamide; other types of anticancer agents such as asparaginase, rituximab, trastuzumab, imatinib, retinoic acid and derivatives, colony-stimulating factors, amifostine, camptothecin, topotecan, thalidomide analogs such as lenalidomide, CDK inhibitor and other HDAC inhibitor such as histone deacetylase 1 inhibitors, histone deacetylase 2 inhibitors, histone deacetylase 3 inhibitors, histone deacetylase 4 inhibitors, histone deacetylase 5 inhibitors, histone deacetylase 6 inhibitors, histone deacetylase 7 inhibitors, histone deacetylase 8 inhibitors, histone deacetylase 9 inhibitors, histone deacetylase 10 inhibitors, and histone deacetylase 11 inhibitors.

Yet another aspect of the present disclosure is to provide a method of inhibiting or treating diseases arising from abnormal cell proliferation and/or differentiation in animal, comprising administering to said animal a therapeutically effective amount of one or more compounds according to the present invention. In one embodiment, the method of inhibiting or treating disease comprises administering to an animal a composition comprising an effective amount of one or more compounds of the invention and a pharmaceutically-acceptable carrier. The composition to be administered may further contain a therapeutic agent such as anti-cancer agent.

A method of the present disclosure is particularly suitable for use with humans, but may be used with other animals, particularly mammals, such as, for example, non-human primates, companion animals, farm animals, laboratory animals, and wild and zoo animals.

A method of the present disclosure is particularly useful to treat diseases mediated directly or indirectly by HDAC since the compounds of the present invention have inhibitory activity against those molecules. In some embodiments, therefore, a method of the present invention is used in inhibiting or treating HDAC-mediated diseases. Examples of such disease include, but are not limited to, cell proliferative diseases such as cancer, autosomal dominant disorders such as Huntington's disease, genetic related metabolic disorder such as cystic fibrosis, fibrosis such as liver fibrosis, renal fibrosis, pulmonary fibrosis and skin fibrosis, autoimmune diseases such as Rheumatoid arthritis, diabetes, acute and chronic neurological diseases such as stroke, amyotrophic lateral sclerosis, hypertrophy such as cardiac hypertrophy, heart failure (or congestive heart failure), and Alzheimer's disease.

In an embodiment, a method according to the present disclosure is applied to a patient with cancer, cystic fibrosis, or pulmonary fibrosis. In some embodiments, a method using a compound according to the present invention is used to treat or inhibit a cancer selected from bladder cancer, breast cancer, colon and rectal cancer, endometrial cancer, kidney (renal cell) cancer, leukemia, lung cancer, melanoma, non-Hodgkin's lymphoma, pancreatic cancer, prostate cancer, skin cancer (non-melanoma), and thyroid cancer.

### EXAMPLES

The following examples are merely illustrative, and do not limit this disclosure in any way.

### Example 1

### N-(2-aminophenyl)-4-[2-(3-dimethylaminomethyl benzofuran-2-yl carbonylamino)-ethoxy]-benzamide

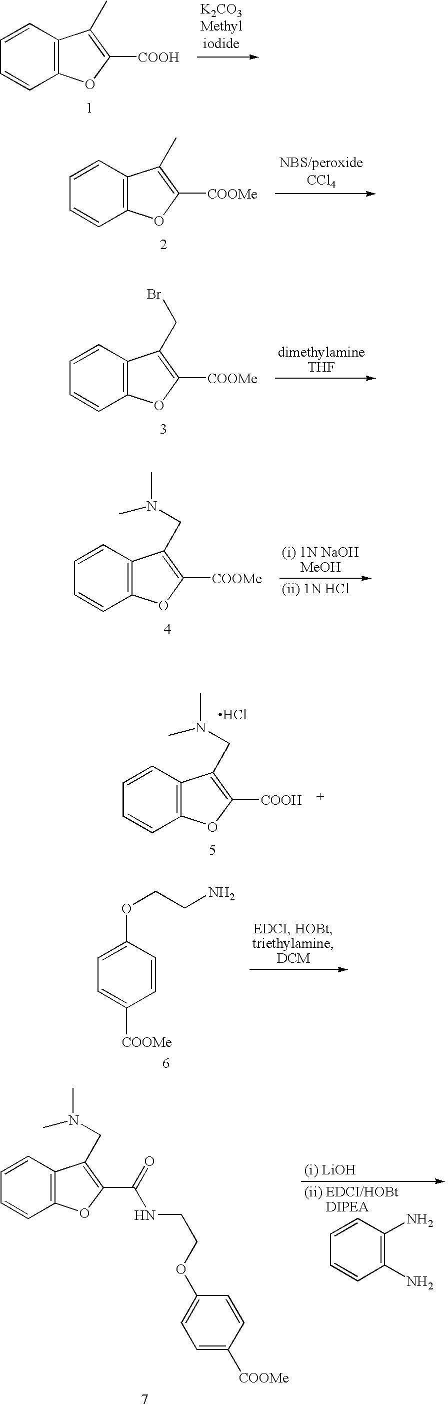

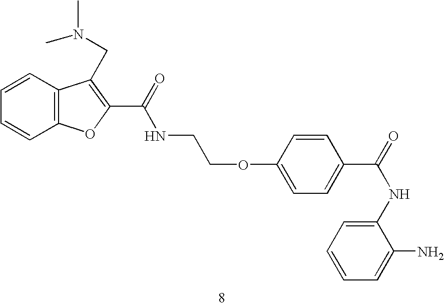

Step (a): To a solution of 3-methyl benzofuran-2-carboxylic acid, starting material 1 (250 mg, 1.42 mmol) in acetone (5 mL) was added potassium carbonate (293 mg, 2.13 mmol) at room temperature followed by methyl iodide (0.10 mL, 1.70 mmol). The reaction mixture was stirred for overnight at room temperature and filtered. The filtrate was concentrated in vacuum and the crude mass was purified by column chromatography using EA:Hexane to produce Intermediate 2.

Step (b): To a solution of Intermediate 2 of step (a) (1 g, 5.3 mmol) in carbon tetrachloride (40 mL) was added N-bromosuccinimide (NBS) (950 mg, 5.3 mmol) and benzoyl peroxide (128 mg, 0.53 mmol) at room temperature. The reaction mixture was refluxed for 4 hours and volatiles were distilled off. The residue was diluted with ethyl acetate (100 mL) and washed with water. The organic layer was dried over anhydrous Na₂SO₄, filtered, and concentrated under vacuum. The residue was triturated with n-hexane (10 mL) and filtered which furnished Intermediate 3.

Step (c): Dimethyl amine gas, by heating of aqueous 40% dimethyl amine solution, was purged into a solution of Intermediate 3 of step (b) (500 mg, 1.85 mmol) in tetrahydrofuran (25 mL) at −5° C. Progress of the reaction was monitored by TLC. After disappearance of the starting material, the reaction mixture was concentrated under vacuum. The crude mass was diluted with ethyl acetate (50 mL) and washed with water (25 mL). The organic layer was dried over anhydrous Na₂SO₄, concentrated and purified by column chromatography to provide Intermediate 4.

Step (d): To a solution of Intermediate 4 of step (c) (5 g, 21.46 mmol) in methanol (50 mL) was added 1N NaOH (25 mL) at room temperature. The reaction mixture was stirred for 4 hours and the pH was adjusted to 3 with 1N HCl at 5° C. The reaction mixture was concentrated under vacuum and the crude was co-distilled with methanol twice to afford Intermediate 5.

Step (e): To Intermediate 5 of step (d) (2 g, 7.82 mmol) in dichloromethane (20 mL) was added 1-ethyl-3-(3′-dimethylaminopropyl)carbodiimide (EDCI) (2.1 g, 10.95 mmol), 1-hydroxybenzotriazole (HOBO (1.26 g, 9.39 mmol) and triethylamine (1.7 g, 101.1 mmol) at 0° C. followed by Compound 6 (1.52 g, 7.82 mmol) at 0° C. The reaction mixture was stirred overnight at room temperature and diluted with water. The organic layer was separated and washed with water. The organic layer was dried over anhydrous Na₂SO₄, and concentrated under vacuum. The residue was purified through column chromatography using MeOH:dichloromethane (DCM) (2:8) to afford Intermediate 7.

Step (f): To Intermediate 7 of step (e) (1.4 g, 3.53 mmol) in a mixture of methanol (10 mL), tetrahydrofuran (THF) (10 mL) and water (10 mL) was added LiOH, H₂O (370 mg, 8.83 mmol) at room temperature and stirred overnight. The reaction mixture was concentrated under vacuum and diluted with water. The pH was adjusted to 6 with 1N HCl and stirred for 15 minutes. The precipitated solids were filtered and dried under vacuum to afford a pre-final acid.

Step (g): To a solution of the pre-final acid of step (f) (900 mg, 2.35 mmol) in 2,5-dimethylfuran (DMF) (10 mL) was added EDCI (1.03 g, 5.37 mmol), HOBt (318 mg, 2.35 mmol) and diisopropyl ethylamine (DIPEA) (753 mg, 1.05 mL, 5.84 mmol) at 0° C. followed by benzene-1,2-diamine (484 mg, 4.47 mmol) at 0° C. The reaction mixture was stirred for overnight at room temperature, diluted with water and stirred for 30 minutes. The precipitated solids were filtered off and washed with water. The washed solid was dried and purified by column chromatography using MeOH:DCM as eluent (2:8), which produced N-(2-aminophenyl)-4-[2-(3-dimethylaminomethyl benzofuran-2-yl carbonylamino)-ethoxy]-benzamide, Compound 8.

¹HNMR: D₆-DMSO; δ 2.20 (s, 6H), 3.72 (m, 2H), 3.82 (s, 2H), 4.25 (t, J=8 Hz, 2H), 4.87 (s, 2H), 6.58 (t, J=8 Hz, 1H), 6.76 (t, J=8 Hz, 1H), 6.96 (t, J=8 Hz, 1H), 7.11 (m, 3H), 7.33 (t, J=8 Hz, 1H), 7.46 (t, J=8 Hz, 1H), 7.64 (d, J=8 Hz, 1H), 7.86 (d, J=8 Hz, 1H), 7.97 (d, J=8 Hz, 2H), 9.54 (s, 1H) and 10.12 (bs, 1H).

¹³CNMR: D₆-DMSO; ppm 38.459, 44.297, 51.214, 66.421, 111.639, 113.934, 116.149, 116.279, 120.010, 121.506, 123.325, 123.574, 126.310, 126.650, 126.832, 126.931, 128.381, 129.706, 143.122, 145.806, 152.912, 159.181, 160.866 and 164.668.

MS: m/z 472.9 (M⁺+1); IR: (KBr, cm⁻¹) 1652 and 1604; MP: 205.1° C.

### Example 2

### Biological Assays

HDAC inhibitory activity of the compound of Example 1 was measured by two types of assays in which HDAC 1 and 6 were used as a target molecule. The first assay was carried out without preincubation after addition of the enzyme. The test compound was suspended in and titrated in DMSO. It was then spotted into a 384-well test plate. The enzyme, HDAC 1 or 6, was diluted in assay buffer containing 25 mM Tris-HCl (pH 8.0), 137 mM NaCl, 2.7 mM KCl, and 0.01% Tween-20 and added to the pre-spotted compound. The peptide substrate containing a fluorophore/quencher pair was diluted in the same assay buffer and added to the compound/enzyme mix initiating the reaction. The reaction incubated at room temperature for about 45 minutes. A concentrated developer solution was diluted in the assay buffer, and added to the reaction. The reaction was incubated at room temperature for about 15 minutes and relative fluorescence was read on an instrument reader.

The second assay is similar to the first assay described above, except that preincubation is carried out for about 3 hours after the enzyme is introduced. The test compound was suspended in, and titrated in DMSO. It was then spotted into a 384-well test plate. The enzyme, HDAC 1 or 6, was diluted in the same assay buffer as used in the previous assay and added to the pre-spotted compound. The enzyme/compound mix was incubated at room temperature for about 3 hours. The peptide substrate containing a fluorophore/quencher pair was diluted in the assay buffer and added to the compound/enzyme mix initiating the reaction. The reaction incubated at room temperature for 45 minutes. A concentrated developer solution was diluted in the assay buffer, and added to the reaction. The reaction was incubated at room temperature for about 15 minutes and relative fluorescence was read on an instrument reader.

The following table shows IC₅₀ data for the compound tested with the protocols described above.

TABLE 1
IC₅₀ of HDAC inhibitor compound

| | HDAC 1 inhibitory activity | | HDAC 6 inhibitory activity | |
|---|---|---|---|---|
| | (IC₅₀ [μM]) | | (IC₅₀ [μM]) | |
| | No | 3-hour | No | 3-hour |
| Compound | preincubation | preincubation | preincubation | preincubation |
| Compound of Example 1 | 0.03 | 0.0033 | >50 | >50 |

The results indicate that the compound has selectivity for class I HDAC over class II HDAC and thus can be useful to treat or inhibit diseases caused by abnormal activities of class I HDAC.

All patents and publications cited herein are incorporated by reference into this application in their entirety.

The words “comprise”, “comprises”, and “comprising” are to be interpreted inclusively rather than exclusively.

What is claimed is:

1. A compound of Formula (I) or a pharmaceutically acceptable salt thereof: 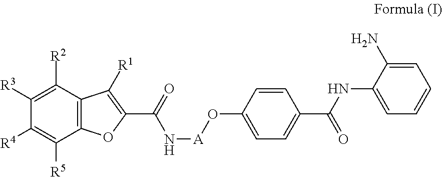
wherein R¹ is selected from the group consisting of H, alkyl, cycloalkyl, alkoxy, amino, aminoalkyl and aryl; and if R¹ is not H, R¹ is optionally substituted with hydroxyl, alkyl, cycloalkyl, alkoxy, amino, aryl or heterocycloalkyl;
wherein R², R³, R⁴ and R⁵ are independently selected from the group consisting of H, alkyl, cycloalkyl, alkoxy, amino, aminoalkyl, aminoalkoxy, carboxyalkylaminoalkyl and aryl; and if any of R², R³, R⁴ and R⁵ is not H, then each of R², R³, R⁴ and R⁵ is optionally substituted with hydroxyl, alkyl, cycloalkyl, alkoxy, amino, aryl, heteroaryl or heterocycloalkyl; and
wherein A is C₂-C₆ alkylenyl optionally substituted with hydroxyl or alkyl, or C₄-C₆ alkenylenyl optionally substituted with alkyl.

2. The compound according to claim 1, wherein for an aryl-containing-substituent of R¹, R², R³, R⁴ and R⁵, aryl is phenyl;
wherein for a heteroaryl-containing-substituent of R¹, R², R³, R⁴ and R⁵, heteroaryl is selected from the group consisting of pyridinyl, pyranyl and imidazolyl; and
wherein for a heterocycloalkyl-containing substituent of R¹, R², R³, R⁴ and R⁵, heterocycloalkyl is selected from the group consisting of pyrrolidinyl, piperidinyl, thiazinyl and morpholinyl.

3. The compound according to claim 1, wherein R¹ is selected from the group consisting of H, methyl, ethyl, propyl, methoxy, ethoxy, methoxymethyl, ethoxyethyl, propoxyethyl, methoxyethoxy, hydroxyethoxy, dimethylamino, diethylamino, dimethylaminomethyl, diethylaminomethyl, dimethylaminoethoxy, benzyl, phenylethyl, phenoxymethyl, phenylethylaminomethyl, benzylaminomethyl, morpholinylmethyl, morpholinylethoxy, imidazolylmethyl, triazinylmethyl, piperidinylmethyl, pyridinylmethoxy, methylpiperazinylmethyl, pyrrolidinylmethyl and pyrrolidinylethoxy;
R², R³, R⁴ and R⁵ are independently selected from the group consisting of H, methyl, ethyl, propyl, methoxy, ethoxy, methoxymethyl, ethoxyethyl, propoxyethyl, methoxyethoxy, hydroxyethoxy, dimethylamino, diethylamino, dimethylaminomethyl, diethylaminomethyl, dimethylaminoethoxy, benzyl, phenylethyl, phenoxymethyl, phenylethylaminomethyl, benzylaminomethyl, morpholinylmethyl, morpholinylethoxy, imidazolylmethyl, triazinylmethyl, piperidinylmethyl, pyridinylmethoxy, methylpiperazinylmethyl, pyrrolidinylmethyl and pyrrolidinylethoxy; and
A is a C₂-C₆ alkylenyl linker optionally substituted with hydroxyl or alkyl.

4. The compound according to claim 3, wherein A is an ethylenyl or propylenyl linker, each optionally substituted with methyl or ethyl.

5. The compound according to claim 3 which is selected from the group consisting of:
N-(2-aminophenyl)-4-[2-(benzofuran-2-yl carbonylamino)-ethoxy]-benzamide;
N-(2-aminophenyl)-4-[3-(benzofuran-2-yl carbonylamino)-propoxy]-benzamide;
N-(2-aminophenyl)-4-[2-(3-methyl benzofuran-2-yl carbonylamino)-ethoxy]-benzamide;
N-(2-aminophenyl)-4-[3-(3-methyl benzofuran-2-yl carbonylamino)-propoxy]-benzamide;
N-(2-aminophenyl)-4-[2-(3-ethyl benzofuran-2-yl carbonylamino)-ethoxy]-benzamide;
N-(2-aminophenyl)-4-[2-(3-propyl benzofuran-2-yl carbonylamino)-ethoxy]-benzamide;
N-(2-aminophenyl)-4-[2-(3-methoxy benzofuran-2-yl carbonylamino)-ethoxy]-benzamide;
N-(2-aminophenyl)-4-[3-(3-methoxy benzofuran-2-yl carbonylamino)-propoxy]-benzamide;
N-(2-aminophenyl)-4-[2-(3-ethoxy benzofuran-2-yl carbonylamino)-ethoxy]-benzamide;
N-(2-aminophenyl)-4-[2-(3-methoxymethyl benzofuran-2-yl carbonylamino)-ethoxy]-benzamide;
N-(2-aminophenyl)-4-[3-(3-methoxymethyl benzofuran-2-yl carbonylamino)-propoxy]-benzamide;
N-(2-aminophenyl)-4-[2-(3-ethoxyethyl benzofuran-2-yl carbonylamino)-ethoxy]-benzamide;
N-(2-aminophenyl)-4-[2-(3-propoxyethyl benzofuran-2-yl carbonylamino)-ethoxy]-benzamide;
N-(2-aminophenyl)-4-[2-(3-methoxyethoxy benzofuran-2-yl carbonylamino)-ethoxy]-benzamide;
N-(2-aminophenyl)-4-[3-(3-methoxyethoxy benzofuran-2-yl carbonylamino)-propoxy]-benzamide;
N-(2-aminophenyl)-4-[2-(3-hydroxyethoxy benzofuran-2-yl carbonylamino)-ethoxy]-benzamide;
N-(2-aminophenyl)-4-[2-(3-dimethylamino benzofuran-2-yl carbonylamino)-ethoxy]-benzamide;
N-(2-aminophenyl)-4-[2-(3-diethylamino benzofuran-2-yl carbonylamino)-ethoxy]-benzamide;
N-(2-aminophenyl)-4-[2-(3-dimethylaminomethyl benzofuran-2-yl carbonylamino)-ethoxy]-benzamide;
N-(2-aminophenyl)-4-[2-(3-diethylaminomethyl benzofuran-2-yl carbonylamino)-ethoxy]-benzamide;
N-(2-aminophenyl)-4-[2-(3-dimethylaminoethoxy benzofuran-2-yl carbonylamino)-ethoxy]-benzamide;
N-(2-aminophenyl)-4-[2-(3-phenylethyl benzofuran-2-yl carbonylamino)-ethoxy]-benzamide;
N-(2-aminophenyl)-4-[2-(3-phenoxymethyl benzofuran-2-yl carbonylamino)-ethoxy]-benzamide;
N-(2-aminophenyl)-4-[3-(3-phenoxymethyl benzofuran-2-yl carbonylamino)-propoxy]-benzamide;
N-(2-aminophenyl)-4-[2-(3-phenylethylaminomethyl benzofuran-2-yl carbonylamino)-ethoxy]-benzamide;
N-(2-aminophenyl)-4-[2-(3-benzylaminomethyl benzofuran-2-yl carbonylamino)-ethoxy]-benzamide;
N-(2-aminophenyl)-4-[2-(3-morpholinylmethyl benzofuran-2-yl carbonylamino)-ethoxy]-benzamide;
N-(2-aminophenyl)-4-[2-(3-morpholinylethoxy benzofuran-2-yl carbonylamino)-ethoxy]-benzamide;
N-(2-aminophenyl)-4-[2-(3-imidazolylmethyl benzofuran-2-yl carbonylamino)-ethoxy]-benzamide;
N-(2-aminophenyl)-4-[2-(3-triazinylmethyl benzofuran-2-yl carbonylamino)-ethoxy]-benzamide;
N-(2-aminophenyl)-4-[2-(3-piperidinylmethyl benzofuran-2-yl carbonylamino)-ethoxy]-benzamide;
N-(2-aminophenyl)-4-[2-(3-pyridinylmethoxy benzofuran-2-yl carbonylamino)-ethoxy]-benzamide;
N-(2-aminophenyl)-4-[2-(3-methylpiperazinylmethyl benzofuran-2-yl carbonylamino)-ethoxy]-benzamide;
N-(2-aminophenyl)-4-[2-(3-pyrrolidinylmethyl benzofuran-2-yl carbonylamino)-ethoxy]-benzamide; and
N-(2-aminophenyl)-4-[2-(3-pyrrolidinylethoxy benzofuran-2-yl carbonylamino)-ethoxy]-benzamide.

6. The compound according to claim 3 which is selected from the group consisting of:
N-(2-aminophenyl)-4-[2-(4-methyl benzofuran-2-yl carbonylamino)-ethoxy]-benzamide;
N-(2-aminophenyl)-4-[3-(4-methyl benzofuran-2-yl carbonylamino)-propoxy]-benzamide;
N-(2-aminophenyl)-4-[2-(4-methyl benzofuran-2-yl carbonylamino)-ethoxy]-benzamide
N-(2-aminophenyl)-4-[2-(4-propyl benzofuran-2-yl carbonylamino)-ethoxy]-benzamide;
N-(2-aminophenyl)-4-[2-(4-methoxy benzofuran-2-yl carbonylamino)-ethoxy]-benzamide;
N-(2-aminophenyl)-4-[3-(4-methoxy benzofuran-2-yl carbonylamino)-propoxy]-benzamide;
N-(2-aminophenyl)-4-[2-(4-ethoxy benzofuran-2-yl carbonylamino)-ethoxy]-benzamide;
N-(2-aminophenyl)-4-[2-(4-methoxymethyl benzofuran-2-yl carbonylamino)-ethoxy]-benzamide;
N-(2-aminophenyl)-4-[3-(4-methoxymethyl benzofuran-2-yl carbonylamino)-propoxy]-benzamide;
N-(2-aminophenyl)-4-[2-(4-ethoxyethyl benzofuran-2-yl carbonylamino)-ethoxy]-benzamide;
N-(2-aminophenyl)-4-[2-(4-propoxyethyl benzofuran-2-yl carbonylamino)-ethoxy]-benzamide;
N-(2-aminophenyl)-4-[2-(4-methoxyethoxy benzofuran-2-yl carbonylamino)-ethoxy]-benzamide;
N-(2-aminophenyl)-4-[3-(4-methoxyethoxy benzofuran-2-yl carbonylamino)-propoxy]-benzamide;
N-(2-aminophenyl)-4-[2-(4-hydroxyethoxy benzofuran-2-yl carbonylamino)-ethoxy]benzamide;
N-(2-aminophenyl)-4-[2-(4-dimethylamino benzofuran-2-yl carbonylamino)-ethoxy]-benzamide;
N-(2-aminophenyl)-4-[2-(4-diethylamino benzofuran-2-yl carbonylamino)-ethoxy]-benzamide;
N-(2-aminophenyl)-4-[2-(4-dimethylaminomethyl benzofuran-2-yl carbonylamino)-ethoxy]-benzamide;
N-(2-aminophenyl)-4-[2-(4-diethylaminomethyl benzofuran-2-yl carbonylamino)-ethoxy]-benzamide;
N-(2-aminophenyl)-4-[2-(4-dimethylaminoethoxy benzofuran-2-yl carbonylamino)-ethoxy]-benzamide;
N-(2-aminophenyl)-4-[2-(4-phenylethyl benzofuran-2-yl carbonylamino)-ethoxy]-benzamide;
N-(2-aminophenyl)-4-[2-(4-phenoxymethyl benzofuran-2-yl carbonylamino)-ethoxy]-benzamide;
N-(2-aminophenyl)-4-[3-(4-phenoxymethyl benzofuran-2-yl carbonylamino)-propoxy]-benzamide;
N-(2-aminophenyl)-4-[2-(4-phenylethylaminomethyl benzofuran-2-yl carbonylamino)-ethoxy]-benzamide;
N-(2-aminophenyl)-4-[2-(4-benzylaminomethyl benzofuran-2-yl carbonylamino)-ethoxy]-benzamide;
N-(2-aminophenyl)-4-[2-(4-morpholinylmethyl benzofuran-2-yl carbonylamino)-ethoxy]-benzamide;
N-(2-aminophenyl)-4-[2-(4-morpholinylethoxy benzofuran-2-yl carbonylamino)-ethoxy]-benzamide;
N-(2-aminophenyl)-4-[2-(4-imidazolylmethyl benzofuran-2-yl carbonylamino)-ethoxy]-benzamide;
N-(2-aminophenyl)-4-[2-(4-triazinylmethyl benzofuran-2-yl carbonylamino)-ethoxy]-benzamide;
N-(2-aminophenyl)-4-[2-(4-piperidinylmethyl benzofuran-2-yl carbonylamino)-ethoxy]-benzamide;
N-(2-aminophenyl)-4-[2-(4-pyridinylmethoxy benzofuran-2-yl carbonylamino)-ethoxy]-benzamide;
N-(2-aminophenyl)-4-[2-(4-methylpiperazinylmethyl benzofuran-2-yl carbonylamino)-ethoxy]-benzamide;
N-(2-aminophenyl)-4-[2-(4-pyrrolidinylmethyl benzofuran-2-yl carbonylamino)-ethoxy]-benzamide; and
N-(2-aminophenyl)-4-[2-(4-pyrrolidinylethoxy benzofuran-2-yl carbonylamino)-ethoxy]-benzamide.

7. The compound according to claim 3 which is selected from the group consisting of:
N-(2-aminophenyl)-4-[2-(5-methyl benzofuran-2-yl carbonylamino)-ethoxy]-benzamide;
N-(2-aminophenyl)-4-[3-(5-methyl benzofuran-2-yl carbonylamino)-propoxy]-benzamide;
N-(2-aminophenyl)-4-[2-(5-ethyl benzofuran-2-yl carbonylamino)-ethoxy]-benzamide
N-(2-aminophenyl)-4-[2-(5-propyl benzofuran-2-yl carbonylamino)-ethoxy]-benzamide;
N-(2-aminophenyl)-4-[2-(5-methoxy benzofuran-2-yl carbonylamino)-ethoxy]-benzamide;
N-(2-aminophenyl)-4-[3-(5-methoxy benzofuran-2-yl carbonylamino)-propoxy]-benzamide;
N-(2-aminophenyl)-4-[2-(5-ethoxy benzofuran-2-yl carbonylamino)-ethoxy]-benzamide;
N-(2-aminophenyl)-4-[2-(5-methoxymethyl benzofuran-2-yl carbonylamino)-ethoxy]-benzamide;
N-(2-aminophenyl)-4-[3-(5-methoxymethyl benzofuran-2-yl carbonylamino)-propoxy]-benzamide;
N-(2-aminophenyl)-4-[2-(5-ethoxyethyl benzofuran-2-yl carbonylamino)-ethoxy]-benzamide;
N-(2-aminophenyl)-4-[2-(5-propoxyethyl benzofuran-2-yl carbonylamino)-ethoxy]benzamide;
N-(2-aminophenyl)-4-[2-(5-methoxyethoxy benzofuran-2-yl carbonylamino)-ethoxy]-benzamide;
N-(2-aminophenyl)-4-[2-(5-methoxyethoxy benzofuran-2-yl carbonylamino)-ethoxy]-benzamide;
N-(2-aminophenyl)-4-[2-(5-hydroxyethoxy benzofuran-2-yl carbonylamino)-ethoxy]-benzamide;
N-(2-aminophenyl)-4-[2-(5-dimethylamino benzofuran-2-yl carbonylamino)-ethoxy]-benzamide;
N-(2-aminophenyl)-4-[2-(5-diethylamino benzofuran-2-yl carbonylamino)-ethoxy]-benzamide;
N-(2-aminophenyl)-4-[2-(5-dimethylaminomethyl benzofuran-2-yl carbonylamino)-ethoxy]-benzamide;
N-(2-aminophenyl)-4-[2-(5-diethylaminomethyl benzofuran-2-yl carbonylamino)-ethoxy]-benzamide;
N-(2-aminophenyl)-4-[2-(5-dimethylaminoethoxy benzofuran-2-yl carbonylamino)-ethoxy]-benzamide;
N-(2-aminophenyl)-4-[2-(5-phenylethyl benzofuran-2-yl carbonylamino)-ethoxy]-benzamide;
N-(2-aminophenyl)-4-[2-(5-phenoxymethyl benzofuran-2-yl carbonylamino)-ethoxy]-benzamide;
N-(2-aminophenyl)-4-[3-(5-phenoxymethyl benzofuran-2-yl carbonylamino)-propoxy]-benzamide;
N-(2-aminophenyl)-4-[2-(5-phenylethylaminomethyl benzofuran-2-yl carbonylamino)-ethoxy]-benzamide;
N-(2-aminophenyl)-4-[2-(5-benzylaminomethyl benzofuran-2-yl carbonylamino)-ethoxy]-benzamide;
N-(2-aminophenyl)-4-[2-(5-morpholinylmethyl benzofuran-2-yl carbonylamino)-ethoxy]-benzamide;
N-(2-aminophenyl)-4-[2-(5-morpholinylethoxy benzofuran-2-yl carbonylamino)-ethoxy]-benzamide;
N-(2-aminophenyl)-4-[2-(5-imidazolylmethyl benzofuran-2-yl carbonylamino)-ethoxy]-benzamide;
N-(2-aminophenyl)-4-[2-(5-triazinylmethyl benzofuran-2-yl carbonylamino)-ethoxy]-benzamide;
N-(2-aminophenyl)-4-[2-(5-piperidinylmethyl benzofuran-2-yl carbonylamino)-ethoxy]-benzamide;
N-(2-aminophenyl)-4-[2-(5-pyridinylmethoxy benzofuran-2-yl carbonylamino)-ethoxy]-benzamide;
N-(2-aminophenyl)-4-[2-(5-methylpiperazinylmethyl benzofuran-2-yl carbonylamino)-ethoxy]-benzamide;
N-(2-aminophenyl)-4-[2-(5-pyrrolidinylmethyl benzofuran-2-yl carbonylamino)-ethoxy]-benzamide; and
N-(2-aminophenyl)-4-[2-(5-pyrrolidinylethoxy benzofuran-2-yl carbonylamino)-ethoxy]-benzamide.

8. The compound according to claim 3 which is selected from the group consisting of:
N-(2-aminophenyl)-4-[2-(6-methyl benzofuran-2-yl carbonylamino)-ethoxy]-benzamide;
N-(2-aminophenyl)-4-[3-(6-methyl benzofuran-2-yl carbonylamino)-propoxy]-benzamide;
N-(2-aminophenyl)-4-[2-(6-ethyl benzofuran-2-yl carbonylamino)-ethoxy]-benzamide
N-(2-aminophenyl)-4-[2-(6-propyl benzofuran-2-yl carbonylamino)-ethoxy]-benzamide;
N-(2-aminophenyl)-4-[2-(6-methoxy benzofuran-2-yl carbonylamino)-ethoxy]-benzamide;
N-(2-aminophenyl)-4-[3-(6-methoxy benzofuran-2-yl carbonylamino)-propoxy]-benzamide;
N-(2-aminophenyl)-4-[2-(6-ethoxy benzofuran-2-yl carbonylamino)-ethoxy]-benzamide;
N-(2-aminophenyl)-4-[2-(6-methoxymethyl benzofuran-2-yl carbonylamino)-ethoxy]-benzamide;
N-(2-aminophenyl)-4-[3-(6-methoxymethyl benzofuran-2-yl carbonylamino)-propoxy]-benzamide;
N-(2-aminophenyl)-4-[2-(6-ethoxyethyl benzofuran-2-yl carbonylamino)-ethoxy]-benzamide;
N-(2-aminophenyl)-4-[2-(6-propoxyethyl benzofuran-2-yl carbonylamino)-ethoxy]-benzamide;
N-(2-aminophenyl)-4-[2-(6-methoxyethoxy benzofuran-2-yl carbonylamino)-ethoxy]-benzamide;
N-(2-aminophenyl)-4-[3-(6-methoxyethoxy benzofuran-2-yl carbonylamino)-propoxy]-benzamide;
N-(2-aminophenyl)-4-[2-(6-hydroxyethoxy benzofuran-2-yl carbonylamino)-ethoxy]-benzamide;
N-(2-aminophenyl)-4-[2-(6-dimethylamino benzofuran-2-yl carbonylamino)-ethoxy]-benzamide;
N-(2-aminophenyl)-4-[2-(6-diethylamino benzofuran-2-yl carbonylamino)-ethoxy]-benzamide;
N-(2-aminophenyl)-4-[2-(6-dimethylaminomethyl benzofuran-2-yl carbonylamino)-ethoxy]-benzamide;
N-(2-aminophenyl)-4-[2-(6-diethylaminomethyl benzofuran-2-yl carbonylamino)-ethoxy]-benzamide;
N-(2-aminophenyl)-4-[2-(6-dimethylaminoethoxy benzofuran-2-yl carbonylamino)-ethoxy]-benzamide;
N-(2-aminophenyl)-4-[2-(6-phenylethyl benzofuran-2-yl carbonylamino)-ethoxy]-benzamide;
N-(2-aminophenyl)-4-[2-(6-phenoxymethyl benzofuran-2-yl carbonylamino)-ethoxy]-benzamide;
N-(2-aminophenyl)-4-[3-(6-phenoxymethyl benzofuran-2-yl carbonylamino)-propoxy]-benzamide;
N-(2-aminophenyl)-4-[2-(6-phenylethylaminomethyl benzofuran-2-yl carbonylamino)-ethoxy]-benzamide;
N-(2-aminophenyl)-4-[2-(6-benzylaminomethyl benzofuran-2-yl carbonylamino)-ethoxy]-benzamide;
N-(2-aminophenyl)-4-[2-(6-morpholinylmethyl benzofuran-2-yl carbonylamino)-ethoxy]-benzamide;
N-(2-aminophenyl)-4-[2-(6-morpholinylethoxy benzofuran-2-yl carbonylamino)-ethoxy]-benzamide;
N-(2-aminophenyl)-4-[2-(6-imidazolylmethyl benzofuran-2-yl carbonylamino)-ethoxy]-benzamide;
N-(2-aminophenyl)-4-[2-(6-triazinylmethyl benzofuran-2-yl carbonylamino)-ethoxy]-benzamide;
N-(2-aminophenyl)-4-[2-(6-piperidinylmethyl benzofuran-2-yl carbonylamino)-ethoxy]-benzamide;
N-(2-aminophenyl)-4-[2-(6-pyridinylmethoxy benzofuran-2-yl carbonylamino)-ethoxy]-benzamide;
N-(2-aminophenyl)-4-[2-(6-methylpiperazinylmethyl benzofuran-2-yl carbonylamino)-ethoxy]-benzamide;
N-(2-aminophenyl)-4-[2-(6-pyrrolidinylmethyl benzofuran-2-yl carbonylamino)-ethoxy]-benzamide; and
N-(2-aminophenyl)-4-[2-(6-pyrrolidinylethoxy benzofuran-2-yl carbonylamino)-ethoxy]-benzamide.

9. The compound according to claim 3 which is selected from the group consisting of:
N-(2-aminophenyl)-4-[2-(7-methyl benzofuran-2-yl carbonylamino)-ethoxy]-benzamide;
N-(2-aminophenyl)-4-[3-(7-methyl benzofuran-2-yl carbonylamino)-propoxy]-benzamide;
N-(2-aminophenyl)-4-[2-(7-ethyl benzofuran-2-yl carbonylamino)-ethoxy]-benzamide
N-(2-aminophenyl)-4-[2-(7-propyl benzofuran-2-yl carbonylamino)-ethoxy]-benzamide;
N-(2-aminophenyl)-4-[2-(7-methoxy benzofuran-2-yl carbonylamino)-ethoxy]benzamide;
N-(2-aminophenyl)-4-[3-(7-methoxy benzofuran-2-yl carbonylamino)-propoxy]-benzamide;
N-(2-aminophenyl)-4-[2-(7-ethoxy benzofuran-2-yl carbonylamino)-ethoxy]-benzamide;
N-(2-aminophenyl)-4-[2-(7-methoxymethyl benzofuran-2-yl carbonylamino)-ethoxy]-benzamide;
N-(2-aminophenyl)-4-[3-(7-methoxymethyl benzofuran-2-yl carbonylamino)-propoxy]-benzamide;
N-(2-aminophenyl)-4-[2-(7-ethoxyethyl benzofuran-2-yl carbonylamino)-ethoxy]-benzamide;
N-(2-aminophenyl)-4-[2-(7-propoxyethyl benzofuran-2-yl carbonylamino)-ethoxy]-benzamide;
N-(2-aminophenyl)-4-[2-(7-methoxyethoxy benzofuran-2-yl carbonylamino)-ethoxy]-benzamide;
N-(2-aminophenyl)-4-[3-(7-methoxyethoxy benzofuran-2-yl carbonylamino)-propoxy]-benzamide;
N-(2-aminophenyl)-4-[2-(7-hydroxyethoxy benzofuran-2-yl carbonylamino)-ethoxy]-benzamide;
N-(2-aminophenyl)-4-[2-(7-dimethylamino benzofuran-2-yl carbonylamino)-ethoxy]-benzamide;
N-(2-aminophenyl)-4-[2-(7-diethylamino benzofuran-2-yl carbonylamino)-ethoxy]-benzamide;
N-(2-aminophenyl)-4-[2-(7-dimethylaminomethyl benzofuran-2-yl carbonylamino)-ethoxy]-benzamide;
N-(2-aminophenyl)-4-[2-(7-diethylaminomethyl benzofuran-2-yl carbonylamino)-ethoxy]-benzamide;
N-(2-aminophenyl)-4-[2-(7-dimethylaminoethoxy benzofuran-2-yl carbonylamino)-ethoxy]-benzamide;
N-(2-aminophenyl)-4-[2-(7-phenylethyl benzofuran-2-yl carbonylamino)-ethoxy]-benzamide;
N-(2-aminophenyl)-4-[2-(7-phenoxymethyl benzofuran-2-yl carbonylamino)-ethoxy]-benzamide;
N-(2-aminophenyl)-4-[2-(7-phenylethylaminomethyl benzofuran-2-yl carbonylamino)-ethoxy]-benzamide;
N-(2-aminophenyl)-4-[2-(7-benzylaminomethyl benzofuran-2-yl carbonylamino)-ethoxy]-benzamide;
N-(2-aminophenyl)-4-[2-(7-morpholinylmethyl benzofuran-2-yl carbonylamino)-ethoxy]-benzamide;
N-(2-aminophenyl)-4-[2-(7-morpholinylethoxy benzofuran-2-yl carbonylamino)-ethoxy]-benzamide;
N-(2-aminophenyl)-4-[2-(7-imidazolylmethyl benzofuran-2-yl carbonylamino)-ethoxy]-benzamide;
N-(2-aminophenyl)-4-[2-(7-triazinylmethyl benzofuran-2-yl carbonylamino)-ethoxy]-benzamide;
N-(2-aminophenyl)-4-[2-(7-piperidinylmethyl benzofuran-2-yl carbonylamino)-ethoxy]-benzamide;
N-(2-aminophenyl)-4-[2-(7-pyridinylmethoxy benzofuran-2-yl carbonylamino)-ethoxy]-benzamide;
N-(2-aminophenyl)-4-[2-(7-methylpiperazinylmethyl benzofuran-2-yl carbonylamino)-ethoxy]-benzamide;
N-(2-aminophenyl)-4-[2-(7-pyrrolidinylmethyl benzofuran-2-yl carbonylamino)-ethoxy]-benzamide; and
N-(2-aminophenyl)-4-[2-(7-pyrrolidinylethoxy benzofuran-2-yl carbonylamino)-ethoxy]-benzamide.

10. The compound according to claim 5 which is

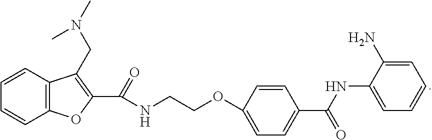

11. A pharmaceutical composition comprising an effective amount of one or more compounds according to claim 1 and a pharmaceutically-acceptable carrier.

12. The pharmaceutical composition according to claim 11, further comprising one or more anti-cancer agents.

13. The pharmaceutical composition according to claim 12, wherein the one or more anti-cancer agents are selected from the group consisting of cyclophosphamide, dacarbazine, cisplatin, methotrexate, mercaptopurine, thioguanine, fluorouracil, cytarabine, vinblastine, paclitaxel, doxorubicin, bleomycin, mitomycin, prednisone, tamoxifen, flutamide, asparaginase, rituximab, trastuzumab, imatinib, retinoic acid, colony-stimulating factor, amifostine, lenalidomide, HDAC inhibitor, CDK inhibitor, camptothecin and topotecan.

14. A method of inhibiting or treating a disease arising from abnormal cell proliferation and differentiation in animal, comprising administering to said animal a therapeutically effective amount of one or more compounds according to claim 1.

15. The method according to claim 14, wherein the animal is human.

16. The method according to claim 15, wherein the disease is mediated by HDAC.

17. The method according to claim 16, wherein the disease is selected from the group consisting of a cell proliferative disease, autosomal dominant disorder, genetic related metabolic disorder, fibrosis, autoimmune disease, cardiac hypertrophy, heart failure, diabetes, neurological disease and Alzheimer's disease.

18. The method according to claim 17, wherein the disease is cancer or pulmonary fibrosis.

19. The method according to claim 18, wherein the cancer disease is selected from the group consisting of bladder cancer, breast cancer, colon and rectal cancer, endometrial cancer, kidney cancer, leukemia, lung cancer, melanoma, non-Hodgkin's lymphoma, pancreatic cancer, prostate cancer, skin cancer and thyroid cancer.
